(19) United States
(12) Patent Application Publication
(10) Pub. No.: US 2024/0057595 A1
(43) Pub. Date: Feb. 22, 2024

(54) INSECT REPELLENT COMPOSITIONS

(71) Applicant: Manus Bio Inc., Waltham, MA (US)

(72) Inventors: Ajikumar Parayil KUMARAN, Waltham, MA (US); Kumar VEDANTAM, Waltham, MA (US)

(21) Appl. No.: 18/270,285

(22) PCT Filed: Dec. 29, 2021
(86) PCT No.: PCT/US21/65446

§ 371 Date: Jun. 29, 2023

Related U.S. Application Data
(60) Provisional application No. 63/131,404, filed on Dec. 29, 2020.

(51) Int. Cl. A01N 35/06; A01N 27/00; A01N 31/06; A01P 17/00; A01N 43/24; A01N 37/18; A01N 37/42; A01N 65/28

(52) U.S. Cl. CPC A01N 35/06; A01N 27/00; A01N 31/06; A01P 17/00; A01N 43/24; A01N 37/18; A01N 37/42; A01N 65/28

(57) ABSTRACT

The present disclosure provides insect and/or arachnid repellent compositions, and methods of use thereof. The compositions disclosed herein comprise nootkatone and one or more fragrance agents and/or non-fragrance agents effective to for repelling the insects or arachnids. The fragrance or non-fragrance agents in various embodiments alter or mask human sweat odor, are effective to repel insects or arachnids, and/or alter or inhibit insect or arachnid olfactory systems.

### PRIORITY

This application claims the benefit of U.S. Provisional Application No. 63/131,404, filed Dec. 29, 2020, the entire disclosure of which is hereby incorporated by reference in its entirety.

### BACKGROUND

Various insects and arachnids cause health and economic hazards by infestation of human and animal bodies (e.g. ticks, mites, and lice) or dwellings (e.g. flies, ants, termites and cockroaches). Some insects act as vectors that transmit human and animal diseases. For example, the deer tick (Ixodes scapularis), an arachnid, transmits the bacterium Borrelia burgdorferi, which causes lyme disease. Mosquitoes (e.g. Aedes aegypti, Aedes albopictus, Culex and Anopheles) spread diseases such as malaria, chikungunya, dengue fever, yellow fever, eastern equine encephalitis, Japanese encephalitis and filariasis to a host on which it feeds. The rat flea (Xenopsylla cheopis) is a vector for Yersinia pestis, the causative agent of bubonic plague.

Pest control is often difficult to achieve or maintain, partly because many available products are toxic to humans, animals or plants, or are pollutants. The U.S. Centers for Disease Control (CDC) recommends DEET (N,N-diethyl-m-toluamide or N,N-diethyl-3-methyl-benzamide), picaridin (icaridin, KBR 3023, 2-(2-hydroxyethyl)-1-piperidinecarboxylic acid 1-methylpropyl ester), oil of lemon eucalyptus (para-menthane-diol or PMD, para-menthane-3,8-diol), IR3535 (3-[N-butyl-N-acetyl]-aminopropionic acid, ethyl ester) and 2-undecanone (methyl nonyl ketone). All have certain side effects and their relative efficacies differ between insect species. Roey et al., Field evaluation of picaridin repellents reveals differences in repellent sensitivity between Southeast Asian vectors of malaria and arboviruses. PLoS Negl Trop Dis 8(12):e3326 (2014). Moreover, mosquitoes become resistant to them. Stanczyk et al., Behavioral insensitivity to DEET in Aedes aegypti is a genetically determined trait residing in changes in sensillum function. Proc. Natl. Acad. Sci. USA 107(19): 8575-8580 (2010).

Therefore, a need exists for repellent compositions capable of controlling a variety of pests, including vectors of disease, and which are safe and effective for use on or around humans, animals, and plants.

### BRIEF DESCRIPTION OF THE DRAWINGS

FIG. 1 shows images of the feeding assay setup. A Hemotek membrane feeding system is loaded with about 3.5-4 ml of bovine blood that is placed on top of a mosquito cage. Treatments to be tested are sprayed on a 7.6×7.6 cm piece of cotton fabric that is placed between the feeding system and the cage.

FIG. 2 is a graph showing the maximum protection time of various nootkatone (“NKT”) solutions.

FIG. 3 is a graph showing the maximum protection time of various NKT solutions.

FIG. 4 is a graph showing mosquito survival during exposure to various solutions.

FIG. 5 shows a schematic and an image of the Tick Carousel Assay.

FIG. 6 shows an image of a control cloth test. Shown in the figure are adult Amblyomma americanum ticks engaged to a control cloth and the tick-infested tick island surrounded by water.

FIG. 7 is a graph showing repellent efficacy of various NKT formulations and controls at initial application.

FIG. 8 is a graph showing repellent efficacy of various NKT formulations and controls at 3 hours post-application.

FIG. 9 is a graph showing repellent efficacy of various NKT formulations and controls at 6 hours post-application.

### DETAILED DESCRIPTION

The present disclosure provides insect and/or arachnid repellent compositions, and methods of use thereof. The compositions disclosed herein comprise the ketone 5,6-dimethyl-8-isopropenyl-bicyclo[4.4.0]dec-1-en-3-one, referred to as nootkatone, and its aldehyde and alcohol analogues, and one or more fragrance agents and/or non-fragrance agents effective for repelling insects or arachnids. The fragrance or non-fragrance agents in various embodiments alter or mask human sweat odor, and/or alter or inhibit insect or arachnid olfactory systems. In some embodiments, the fragrance or non-fragrance agents have an activity of repelling insects or arachnids, and are synergistic with nootkatone.

Insects, including mosquitos, and lyme disease ticks, use their sense of smell for long range host seeking. Key proteins involved in insect olfaction include odorant receptors, odorant receptor co-receptors, gustatory receptors, and odorant binding proteins. Various mosquito attractants include odorous molecules produced by human metabolism or the bacterial degradation of sweat components, lactic acid, fatty acids, steroids on skin, and carbon dioxide that is exhaled. All these molecules act as strong attractants for mosquitos. Since this attraction is based on the sense of smell of mosquitos, compositions can be designed or engineered to block/confuse this sense of smell.

Without being bound by theory, it is believed that the compositions disclosed herein will work by one or more mechanisms (including with synergistic active agents) to: alter the smell of human sweat components, mask human sweat components, impact the trigeminal system of insects thereby altering the insect's sense of smell, and have molecules that sublime and physically block insect olfactory receptors. The present disclosure is based, in part, on the discovery that compositions or consumer products comprising nootkatone (a component of grapefruit oil) in combination with fragrant and non-fragrant agents (including perfume materials) are effective insect or arachnid repellents. As disclosed herein, certain perfumery and non-perfumery materials can be formulated with nootkatone to create compositions that eliminate and/or reduce host-vector interactions in relation to insects seeking hosts and provides protection to consumers from insect bites. The compositions described herein are generally safe for external use on humans and animals.

Sesquiterpenoids of the following type belong to the nootkatone family: 4-α,5-Dimethyl-1,2,3,4,4a,5,6,7-octahydro-7-keto-3-isopropenylnaphthalene. In some embodiments, the nootkatone-containing ingredient is as described in U.S. Pat. No. 10,501,760 or U.S. Pat. No. 10,934,564, which are hereby incorporated by reference in their entireties. In various embodiments, the nootkatone-containing ingredient includes oxygenated valencene, and which is at least about 40% nootkatone, or at least about 50% nootkatone, or at least about 70% nootkatone, or at least about 80% nootkatone, or at least about 90% nootkatone. In some embodiments, the composition further comprises one or more of valencene, β-nootkatol, and α-nootkatol.

In exemplary embodiments, the composition comprises at least about 2% nootkatone (by weight), or at least about 2.5% nootkatone, or at least about 3% nootkatone, 3.5% nootkatone, or at least about 4% nootkatone, or at least about 5% nootkatone by weight. In various embodiments, the composition comprises at least 0.25%, or at least about 0.5%, or at least about 1.0% by weight of other terpenes and terpenoids, including but not limited to sesquiterpenoids. Exemplary sesquiterpenoids comprise valencene and nootkatol (α- and/or β-nootkatol). Other terpenoid compounds that may be employed are described elsewhere herein. In various embodiments, the composition comprises from about 2% to about 10% nootkatone, or from about 2% to about 8% nootkatone, or from about 2% to about 6% nootkatone. In some embodiments, the composition comprises from about 3% to about 10% nootkatone, or from about 3% to about 8% nootkatone, or from about 3% to about 6% nootkatone. In various embodiments, the composition comprises about 4%, about 4.5%, or about 5% nootkatone, optionally with from about 1% to about 2% of additional terpenes or terpenoids (including but not limited to sesquiterpenes and sesquiterpenoids, such as valencene and nootkatol).

In various embodiments, the one or more of the fragrance agents and/or non-fragrance agents are sublimable. In these embodiments, the substance has a high vapor pressure and thus the molecules can easily escape into the atmosphere. For example, in various embodiments, about 10% to about 90% of the composition may be sublimable. In some embodiments, at least about 20%, or at least about 40%, or at least about 60%, or at least 70%, or at least 80% of the composition is sublimable. In various embodiments, one or more of the fragrance agents have an average molecular weight of about 300 Da or less, or an average molecular weight of about 250 Da or less, or an average molecular weight of about 225 Da or less.

In some embodiments, the fragrance agent has an odor detection threshold (ODT) less than about 0.5 ppb, or has an ODT of less than about 0.2 ppb, or less than about 0.1 ppb. The odor detection threshold is the lowest concentration of a certain odor compound that is perceivable by the human sense of smell. In some embodiments, the fragrance agent has an ODT of from 0.0001 ppb to about 0.5 ppb, or from about 0.001 to about 0.1, or from about 0.01 to about 0.1 ppb.

In various embodiments, one or more of the fragrance agents comprise terpenoid compounds. Exemplary terpenoid compounds are monoterpenoids, diterpenoids, or sesquiterpenoids. Exemplary terpenoid compounds that may be included can be selected from 1,8-cineol, anisole, anethole, eugenol, thymol, linalool, borneol, citronellol, menthol, isomenthol, camphor, camphene, carvone, geraniol, menthone, pulegone, limonene, α-pinene, β-pinene, sabinene, myrcene, α-terpinene, β-bisabolene, trans-α-bergamotene, nerol, neral, cedrol, cedrene, thujopsene, methyl thujate, and thujic acid. In some embodiments, a fragrance agent is selected from menthol (e.g., menthol carboxamide), ethylene brassylate, oil of lemon eucalyptus, camphor, and geraniol.

In some embodiments, the composition comprises menthol and/or menthol carboxamide, which are monoterpenoids with a structure based on the o-, m-, or p-menthane backbone. In various embodiments, menthol and/or menthol carboxamide is present in the composition from 0.1% to about 10% by weight, such as from about 0.1% to about 5% by weight, such as from about 2.5% to about 1% by weight (e.g., about 0.5%).

In some embodiments, the composition comprises ethylene brassylate, also known as astratone or musk T, which is a macrolide having a sweet, ambrette, and floral taste. In various embodiments, the ethylene brassylate is present in the composition from about 0.1% to about 10% by weight, such as from about 0.1% to about 5% by weight, such as from about 2.5% to about 1% by weight (e.g., about 0.5%).

In some embodiments, the composition comprises methyl dihydrojasmanate. Methyl dihydrojasmonate is an aroma compound that smells similar to jasmine. In racemic mixtures the odor is floral and citrus while epimerized mixtures exhibit a dense fatty floral odor with odor recognition thresholds of 15 parts per billion. The compound is also known as hedione. In various embodiments, the methyl dihydrojasmanate is present in the composition from about 0.1% to about 10% by weight, such as from about 0.1% to about 5% by weight, such as from about 2.5% to about 1% by weight (e.g., about 0.5%).

In some embodiments, the composition comprises oil of lemon eucalyptus, which is an essential oil distilled from the leaves of the lemon eucalyptus tree. In various embodiments, oil of lemon eucalyptus is present in the composition from about 1% to about 20% by weight, such as from about 1% to about 10% by weight, such as from about 2.5% to about 7.5% by weight (e.g., about 5% by weight).

In some embodiments, the composition comprises camphor, which is a cyclic monoterpene ketone that is bornane bearing an oxo substituent at position 2. In various embodiments, camphor is present in the composition from about 0.1% to about 10% by weight, such as from about 0.1% to about 5% by weight, such as from about 2.5% to about 1% by weight (e.g., about 0.5% by weight).

In some embodiments, the composition comprises (in addition to nootkatone or nootkatone-containing ingredient) two, three, or four of: menthol or menthol carboxamide, ethylene brassylate, oil of lemon eucalyptus, and camphor. Such compositions may have a weight % of each ingredient as described above.

In these or other embodiments, the composition comprises DEET (N,N-diethyl-meta-toluamide). DEET is used as the active ingredient in some insect or arachnid repellent products. In various embodiments, DEET is employed about 25% or less, such as about 15% or less, or about 10% or less (by weight).

In some embodiments, the composition comprises one or more of soy bean oil (e.g., in the range of about 0.5% to about 10% by weight (e.g., 1% to about 5% by weight, or about 2% to about 5% by weight, or about 2% to about 4% by weight), and menthane 3,8 diol (e.g., in the range of 10% to about 75% by weight, or in the range of about 20% to about 50% by weight).

Other perfume materials and/or terpene or terpenoid compositions which can be employed in various embodiments include musk oil, civet oil, ambergris oil, castoreum oil, abies oil, ajowan oil, almond oil, ambrette seed absolute, angelic root oil, anise oil, basil oil, bay oil, benzoin resinoid, bergamot oil, birch oil, bois de rose oil, broom absolute, cajeput oil, cananga oil, capsicum oil, caraway oil, cardamon oil, carrot seed oil, cassia oil, cedar leaf oil, cedar wood oil, celery seed oil, cinnamon bark oil, citronella oil, clary sage oil, clove oil, cognac oil, coriander oil, cubeb oil, cumin oil, camphor oil, dill oil, elemi gum, estragon oil, eucalyptol nat., eucalyptus oil, fennel sweet oil, galbanum res., garlic oil, geranium oil, ginger oil, grapefruit oil, hop oil, hyacinth absolute, jasmine absolute, juniper berry oil, labdanum res., lavender oil, laurel leaf oil, lemon oil, lemongrass oil, lime oil, lovage oil, mace oil, mandarin oil, mimosa absolute, myrrh absolute, mustard oil, narcissus absolute, neroli bigarade oil, nutmeg oil, oakmoss absolute, olibanum res., onion oil, opoponax res., orange oil, orange flower oil, origanum, orris concrete, pepper oil, peppermint oil, peru balsam, petitgrain oil, pine needle oil, rose absolute, rose oil, rosemary oil, safe officinalis oil, sandalwood oil, sage oil, spearmint oil, styrax oil, thyme oil, tolu balsam, tonka beans absolute, tuberose absolute, turpentine oil, vanilla beans absolute, vetiver oil, violet leaf absolute, ylang oil, α-pinene, β-pinene, d-limonene, 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol, anethole, musk xylol, isoeugenol, methyl eugenol, α-amylcinnamic aldehyde, anisaldehyde, n-butylaldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hydroxylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone, amyl butyrolactone, diphenyl oxide, methyl phenyl glycidate, 7-nonyl lactone, coumarin, cineole, ethyl methyl phenyl glicydate, methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl .alpha.-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, and vetiveryl acetate. In various embodiments, individual ingredients are present in the composition from 0.1% to about 10% by weight, such as from about 0.1% to about 5% by weight, such as from about 2.5% to about 1% by weight (e.g., about 0.5%).

In various embodiments, the composition further comprises one or more of a stabilizer, a preservative, an antioxidant, a solvent, a chelating agent, a surfactant, an antifoaming agent, a viscosity regulator, a binder, a propellant, and an encapsulating agent. In embodiments where the composition comprises encapsulation matrix, the matrix may be water soluble or water insoluble. In general, such components can be any of those components conventionally used for topical compositions, including those disclosed in US 2019/0365629, WO 2006/123324, which are hereby incorporated by reference in their entireties. Exemplary matrices for encapsulation of volatile organic compounds are disclosed in US 2016/0330952, which is hereby incorporated by reference in its entirety. Other encapsulation technologies can include, without limitation, use of polymers, liposomes, and β-cyclodextrin. In exemplary embodiments, the composition can be mixed with a fluid to provide a homogenous mixture with simple shaking to vigorous stirring.

In various embodiments, the composition is packaged as a product selected from a skin care product, a fabric care product, an air care product, a hair care product, a home and/or environment care product, a hygiene/beauty care product, a fine fragrance, an over-the-counter health product, and a device for one or more thereof. In some embodiments, the product is for use on a human body, an animal body (e.g., for pet care), in indoor air, in outdoor air, or on an inanimate surface. In various embodiments, the product is a consumer-packaged good for protecting consumers, pets and their homes from invading insects, pests and similar blood sucking arthropods. These products eliminate and/or reduce host-vector interactions in relation to insects seeking hosts and provides protection to consumers from insect bites and the diseases occur as result of these bites.

In other aspects, this disclosure provides a method of repelling an insect or an arachnid. The method comprises contacting air from a space or contacting a surface with the composition disclosed herein. For example, the composition can be contacted with an indoor space or an outdoor space. In some embodiments, the surface is human skin, human hair, or human clothing; or is an animal skin or animal fur. Inanimate surfaces include fabrics, as well as floors, walls, and other home surfaces including countertops.

Numerous methods to reduce host-vector interactions are available which include elimination/reduction of insect sources using insecticide formulations, creating a physical barrier using nets and screens. An easier and inexpensive and safe way implementable at individual level is application of insect or arachnid repellent formulations on areas of skin exposed to insects. The formulations disclosed herein are especially useful when human activity coincides with the diurnal activity patterns of mosquitoes, e.g., camping, hunting and fishing.

In various embodiments, the composition is effective for repelling insects of the taxonomic order Blattaria, Anoplura, Sternorrhyncha, Auchenorrhyncha, Coleorrhyncha, Hymenoptera, Lepidoptera and Siphonaptera. For example, in various embodiments, the composition is effective for repelling insects selected from mosquitoes, lice, fleas, bed bugs, kissing bugs, ants, bees, wasps, flies, and sand flies, among others. In some embodiments, the composition is effective for repelling arachnids of the taxonomic order Acarina, such as ticks, mites and spiders.

In still other aspects, the invention provides a method of reducing transmission of an infection by an insect or arachnid vector, the method comprising contacting a human or animal subject, or contacting air or surface proximal to said human or animal subject, with the composition disclosed herein. In some embodiments, the infection is selected from an infection caused by an arbovirus, Lyme disease, bubonic plague, malaria, leishmaniasis, onchocerciasis, Chagas Disease, filariasis, Schistosomiasis, Typhus, Crimean-Congo haemorrhagic fever, Sleeping sickness (African trypanosomiasis), Louse-borne relapsing fever, Anaplasmosis, Babesiosis, Borrelia mayonii infection, Borrelia miyamotoi infection, Bourbon virus infection, Colorado tick fever, Ehrlichiosis, Heartland virus infection, Powassan disease, Rickettsia parkeri rickettsiosis, Rocky Mountain spotted fever (RMSF), STARI (Southern tick-associated rash illness), Tickborne relapsing fever (TBRF), Tularemia, and 364D rickettsiosis. In some embodiments, the arbovirus is selected from Dengue virus, Zika virus, Japanese encephalitis virus, Rift Valley fever virus, Tick-borne encephalitis virus, West Nile virus and Yellow fever virus. In some embodiments, the vector is selected from a mosquito, a fly, a Tsetse fly, a sand fly, a louse, a flea, a tick, a mite, and a black fly.

In various embodiments, the composition is contacted with human skin, human hair, or human clothing; or is contacted with animal skin or animal fur. In some embodiments, the surface is an inanimate surface selected from fabric or other home surfaces such as floors, walls, and counters.

### EXAMPLES

The Examples below evaluate different compositions comprising nootkatone (“NKT”), prepared as described previously (see U.S. Pat. No. 10,501,760 or U.S. Pat. No. 10,934,564, which are hereby incorporated by reference in their entireties). The stock compositions contain about 75% nootkatone in ethanol, with other minor terpene compounds. The nootkatone was diluted in different amounts of ethanol, to produce compositions ranging from 0.5% to 5% of nootkatone in ethanol.

### Example 1: Mosquito Contact-Repellency of NKT Dilutions

This example evaluated the contact-repellency of different NKT treatments on female Aedes aegypti mosquitoes. In these experiments, the repellent efficacy of the NKT solution was compared to 0.5% permethrin, and an untreated control.

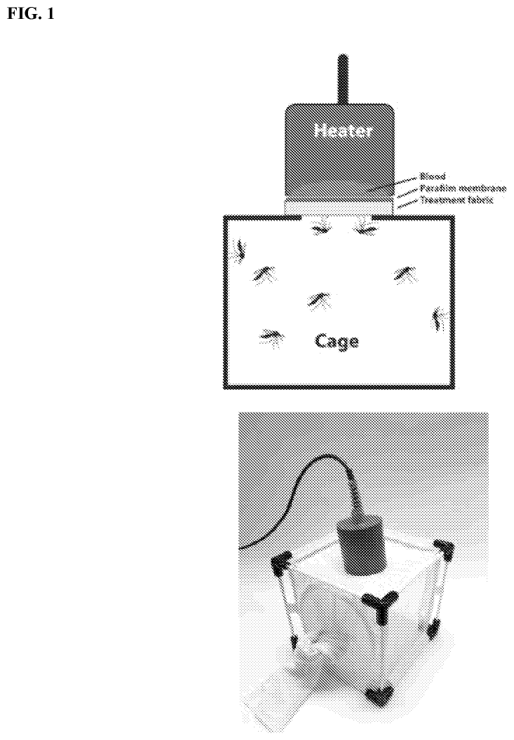

A feeding assay was used to test the strength of contact-repellency of mosquito repellents (FIG. 1). The parts of the feeding assay included: (1) a 17.5×17.5 cm mesh cage, (2) 3.5-4 ml of bovine blood, (3) a Hemotek membrane feeding system set to 37 degrees Celsius, and (4) a treated 7.6×7.6 cm cotton cloth. The experiments were performed in an environmentally controlled room (24-26° C. and 16-26% humidity), and five experimental replicates were performed for each solution/product tested. A water-treated negative control replicate was performed before each experiment in order to test the normal host seeking and feeding behavior of the female mosquitoes. Control replicates (“NKT-0” is a control, 100% ethanol) were performed synchronous with experimental testing of each product. Repellency of products were accessed at initial application and every 15 minutes, up to 72 hours, or until a blood meal was taken. 50 female mosquitoes were used per replicate. All statistical tests were performed using Prism8. P-values less than 0.05 were considered significantly different. The distribution of the data was graphically inspected using quantile-quantile (Q-Q) plots. One-way ANOVA was used to analyze the significant difference in protection time between the treatment groups. Tukey's post hoc test was done for multiple comparisons.

The Aedes aegypti UGAL mosquito strain was used in this example. This mosquito strain is the principal vector of Dengue fever, Chikungunya virus, Zika virus, and yellow fever, and has a worldwide distribution pattern in tropical, subtropical, and temperate climate zones.

In these experiments, mosquitoes were initially starved of water and sugar for 12 hours before the experiments were conducted. Next, 50 female mosquitoes were transferred to a mesh cage, and then a piece of cotton cloth was treated by spraying 350 μl of NKT solution. The cloth was either used immediately or stored on an open counter for a measured amount of time. The treatment (i.e., cloth treated with NKT dilution, DEET, or Permethrin solution) or a control (cloth treated with water or NKT-0) was then placed on top of the mesh of the cage covering the 5 cm diameter circular hole. The prepared Hemotek membrane feeding system with 3.5-4 ml of bovine blood was then placed on top of the experimental or control cloth. The Hemotek membrane feeding system has 6 feeders, which allowed testing 6 cages at a time (5 experimental replicates, and 1 NKT-0 control). Mosquito behavior was then monitored and recorded for the initial 2 minutes, and then every 15 minutes or until a mosquito was engorged. Engorgement with blood (blood feeding) indicated breakdown of contact repellency. If the control mosquitoes did not take a blood meal within the first 2 minutes, the experiment was postponed for 24 hours. Mosquito feeding behavior was monitored for up to 72 hours or until the contact repellency had failed.

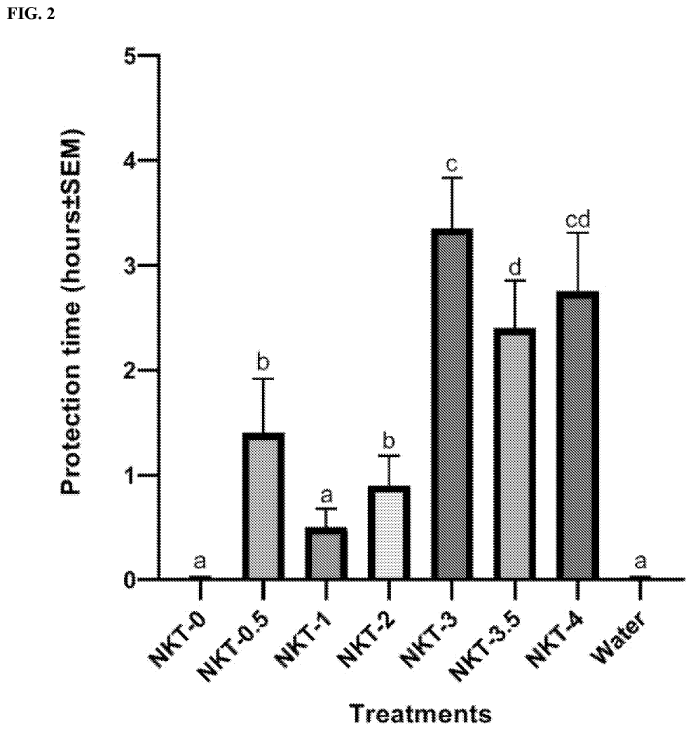

FIG. 2 shows the results of the feeding assay performed in the presence of six experimental treatments and two controls. Each column in FIG. 2 shows the average protection time in hours from mosquito blood feeding under control conditions (columns labeled NKT-0 and water) and in the presence of various NKT solutions. The column letters (a,b,c,d) in FIG. 2 refer to the statistical significance; columns sharing a letter are not significantly different from each other, while columns not sharing a letter are significantly different from each other.

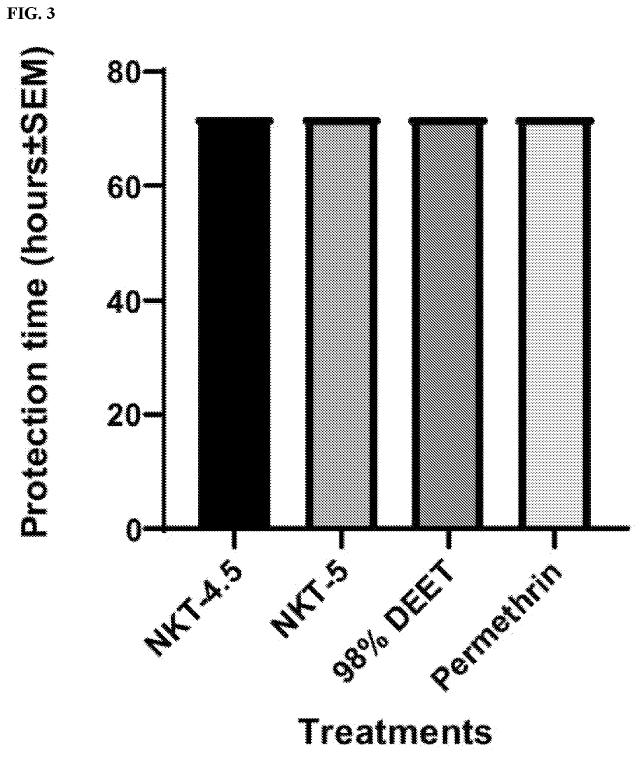

FIG. 3 shows the results of the feeding assay performed in the presence of four experimental treatments. Testing stopped after 72 hours.

These experiments demonstrate that the tested NKT solutions broadly fell into three categories (see FIG. 2). The NKT 0 control did not show any mosquito repellency. The next group with the “NKT 0.5” concentration (about 0.5% of nooktkatone in ethanol), “NKT 1” concentration (about 1% of nooktkatone in ethanol), and “NKT 2” concentration (about 2% of nooktkatone in ethanol) had average maximum protection times between 30 and 90 min. The next group with the “NKT 3” concentration (about 3% ofnooktkatone in ethanol), “NKT 3.5” concentration (about 3.5% of nooktkatone in ethanol), and “NKT 4” concentration (about 4% of nooktkatone in ethanol) had average maximum protection times between two to three hours. The third group, “NKT 4.5” concentration (about 4.5% of nooktkatone in ethanol), and “NKT 5” concentration (about 5% of nooktkatone in ethanol), conferred a protection time of 72 hours, at which time the tests were stopped (see FIG. 3).

In summary, the different NKT dilutions provided protection from mosquito bites in a concentration-dependent distribution pattern. All concentrations tested provided initial mosquito repellency during the tests shown in this example.

### Example 2: Mortality of Mosquitoes Exposed to NKT Dilutions

A mortality assay was employed to evaluate mosquito mortality in response to exposure to eleven different treatments including water, a series of NKT dilutions, and 0.5% permethrin. The Aedes aegypti UGAL mosquito strain (adult female) was used in this example.

The experiments were conducted in a well-lit, temperature- and humidity-controlled room. The room was 22-24° C. with humidity at 40-45%. To conduct these experiments, each treatment had four replicates under the same conditions. A filter paper, 10 cm in diameter, was treated with 340 μl of an experimental or control solution using a standardized spray bottle (113.3 μl/spray) that was held approximately 5 cm away from the paper. The treated filter paper was placed into an open petri dish with a diameter of 10 cm. The treated paper was left in the open petri dish for 60 minutes prior to testing to allow for the evaporation of inactive ingredients. Female mosquitoes were then anesthetized on ice for five minutes. After 60 minutes, 10 ice-anesthetized adult female mosquitoes were transferred onto the treated paper using soft featherweight forceps. The petri dish lid was placed on top, and the mosquitoes were given two minutes to revive before the timer was started. Mosquitoes were then monitored every five minutes. Mosquitoes on their back were considered knocked down and marked as deceased in these experiments. All statistical tests were performed using Prism8, and p-values less than 0.05 were considered significantly different. Log-rank (Mantel-Cox) test was performed for comparison of survival curves.

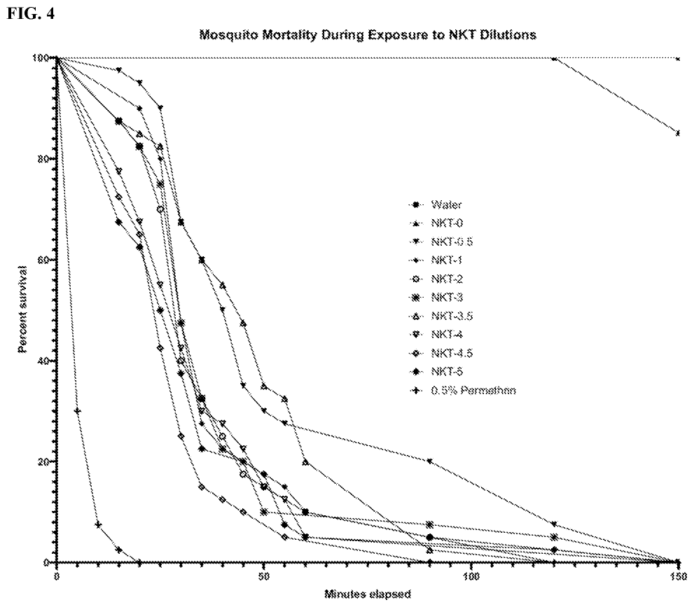

The results in FIG. 4 show the percentage of mosquitoes that were recorded as alive during a 150 min exposure time to various treatments. These experiments show that mosquitoes treated with water or NKT-0 caused either no mortality or few mortality events during the duration of the experiment. The 0.5% permethrin solution had a strong immediate knockdown effect within the first 20 minutes. All NKT dilutions tested resulted in more than 60% knockdown within the first hour, and killed all mosquitoes by the 150 min time point.

In summary, the NKT dilutions tested in the experiments of this example are highly toxic to adult mosquitoes.

### Example 3: Determining Tick Repellency of NKT Formulations Using the Tick Carousel Assay

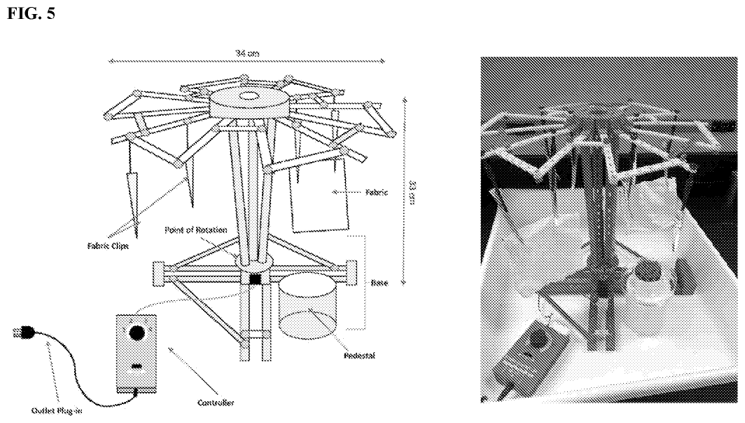

The experiments of this example measured the repellent efficacy of seven NKT formulations and two commercially available repellent products on adult Amblyomma americanum (Lone Star ticks). A Tick Carousel Assay was used (see FIG. 5) to determine the repellent properties of various treatments on adult Amblyomma americanum. A schematic of the Tick Carousel Assay is shown on the left side of FIG. 5, and a photo image of the Tick Carousel Assay is on the right side of FIG. 5.

The Tick Carousel Assay was configured to determine the number of tick engagements to treated and control cloths (FIG. 5). The parts of the Tick Carousel Assay include a carousel rotation element, alligator clips that attach the cloths to the carousel, and a pedestal used to place a tick island. The tick island is made of faux grass, and is in the center of a petri dish that is filled with water to prevent ticks from leaving the tick island. All experiments were conducted in a climate/humidity-controlled room that ranged from 25-26° C., with a relative humidity of 44% to 78%. Four experimental replicates were performed for each formulation, and four negative (untreated) control replicates were performed prior to each experimental replicate. Repellency testing was conducted at initial application, and at three- and six-hours post application to the cloth, and the number of engagements were recorded over 5-minute periods for control and experimental tests. Fourteen lone star ticks were used per replicate.

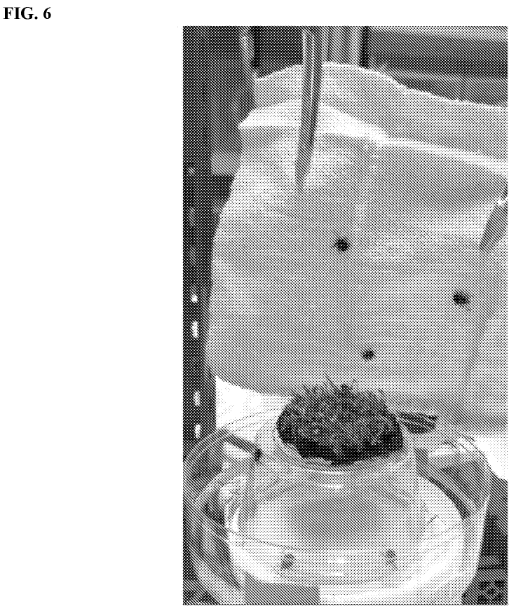

To start the experiment, 14 ticks were placed on the grass portion of the tick island (FIG. 6). Two cloth pieces (11.5×11.5 cm) were then sprayed with 0.5 ml (6 sprays) of test solution from about 4 cm away (˜20 ng/cm²). The treated fabrics were either used immediately or stored at room temperature (sprayed side facing up) for a defined time (3 or 6 hours). The two cloths, either control or sprayed with the treatment, were then clipped onto the Tick Carousel Assay. The experimenter then exhaled air towards the tick island at the beginning of the 5-minute period and again at 2.5 minutes. Once the timer was started, the Tick Carousel Assay was switched on, and the treated cloths brushed over the tick island for 5 minutes (100 passes of the two cloths). During the 5 minutes, the number of tick engagements were tallied using a handheld tally counter. Once the 5 minutes were up, the Tick Carousel Assay was switched off, and the data was recorded. The number of tick engagements for each treatment at each time point was then compared to untreated controls to determine the repellent efficacy of the treatments.

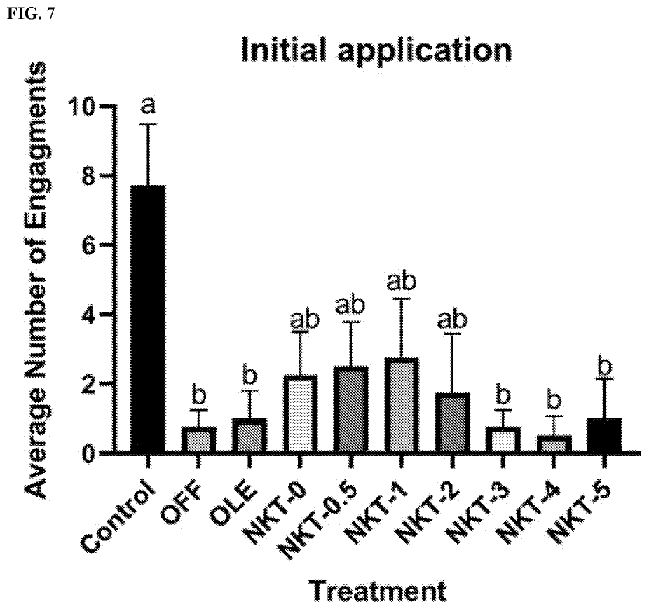

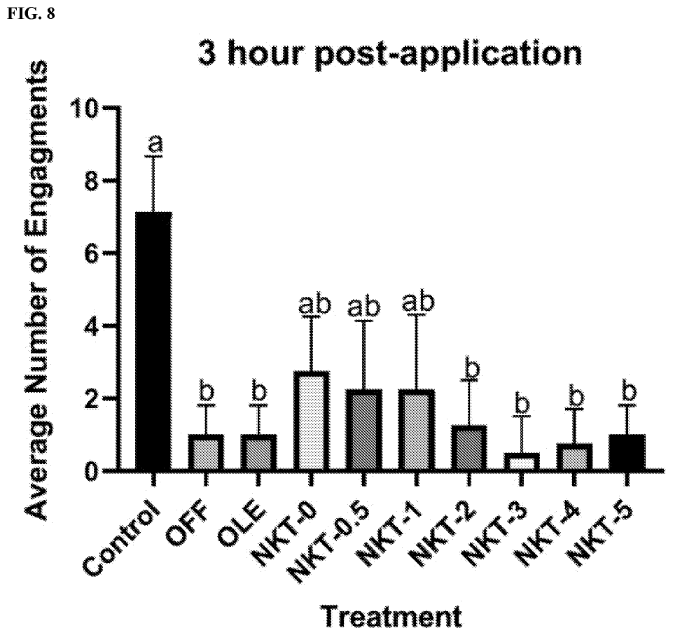

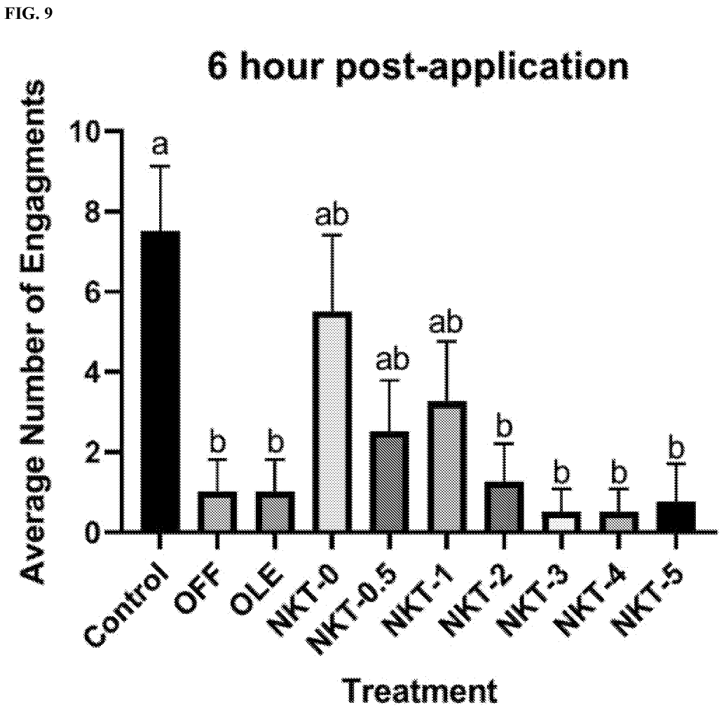

FIG. 7 shows the initial average, FIG. 8 shows the 3-hour post-application, and FIG. 9 shows 6-hour post-application of the number of Amblyomma americanum that engaged to a piece of cloth throughout a 5-minute test period (OFF: 25% DEET. OLE: 30% Oil of Lemon Eucalyptus). The error bars on top of each column in FIG. 7, FIG. 8, and FIG. 9 shows the standard error of that treatment and the letters above each column distinguish groups with significantly different engagement rates (Kruskal-Wallis test was performed to determine statistical significance). Columns that share a letter are statistically not different, while columns that do not share a letter are statistically different.

In summary, of the seven NKT-formulations tested using the Tick Carousel Assay, NKT-0, NKT-0.5, and NKT-1 concentrations had no significant repellency effect on ticks. NKT-3, NKT-4, NKT-5 concentrations, and the positive controls (OFF and OLE) showed strong tick repellency at all three time points tested. The NKT-2 concentration repelled ticks significantly at the 3- and 6-hour time point.

### Example 4. Additional Insect or Arachnid Repellent Composition Disclosed Herein

The following compositions will be prepared for synergistic activity for insect repelling or filling activity:

| No. | Test Candidate | Active Ingredient |
|---|---|---|
| 1 | Nootkatone | 0.1-5% |
| 2 | Nootkatone | 0.1-5% |
| | Menthol carboxamide | 0.1-5% |
| 3 | Nootkatone | 0.1-5% |
| | Ethylene Brassylate | 0.1-2% |
| 4 | Nootkatone | 0.1-5% |
| | Methyl Dihydrojasmanate | 0.1-10% |
| 5 | Nootkatone | 0.1-5% |
| | Menthol Carboxamide | 0.1-2% |
| | Ethylene Brassylate | 0.1-10% |
| 6 | Nootkatone | 0.1-5% |
| | Menthol Carboxamide | 0.1-2% |
| | Ethylene Brassylate | 0.1-2% |
| | Oil of Lemon Eucalyptus | 0.1-5% |
| 7 | Nootkatone | 0.1-5% |
| | Menthol | 0.1-5% |
| | Camphor | 0.1-5% |
| 8 | Nootkatone | 0.1-5% |
| | Menthol | 0.1-2% |
| | Camphor | 0.1-5% |
| | Ethylene Brassylate | 0.1-5% |
| 9 | Nootkatone | 0.1-5% |
| | DEET | 0.1-5% |
| 10 | Nootkatone | 0.1-5% |
| | Menthol Carboxamide | 0.1-2% |
| | Ethylene Brassylate | 0.1-2% |
| | Oil of Lemon Eucalyptus | 0.1-5% |
| | Methyl Dihydrojasmanate | 0.1-10% |
| | Menthol | 0.1-2% |
| | Camphor | 0.1-5% |
| | Oil of Lemon Eucalyptis | 0.1-5% |

It is expected that one or more of compositions 3-9 will repel mosquitoes more efficiently and/or for a longer duration than that for composition 1 and/or composition 2. It is further anticipated that the one or more of compositions 3-9 will be able to repel one or more kinds of insects (e.g. different species of mosquitoes, ticks, etc.) more efficiently and/or for a longer duration than that for composition 1 and/or composition 2.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1. An insect or arachnid repellent composition comprising nootkatone and one or more fragrance agents and/or non-fragrance agents effective to: alter or mask human sweat odor, effective to repel insects or arachnids, and/or alter or inhibit insect or arachnid olfactory system.

2. The composition of claim 1, further comprising one or more of valencene, β-nootkatol, and α-nootkatol.

3. The composition of claim 1 or 2, wherein the nootkatone is present in the composition at about 0.1% to about 10% by weight.

4. The composition of claim 3, wherein the nootkatone is present in the composition at about 0.5% to about 5% by weight.

5. The composition of claim 3, wherein the nootkatone is present in the composition at a concentration of at least about 2% by weight.

6. The composition of claim 5, wherein the nootkatone is present in the composition at a concentration of at least about 4% by weight.

7. The composition of any one of claims 1 to 6, wherein one or more of the fragrance agents and/or non-fragrance agents are sublimable.

8. The composition of claim 7, wherein about 10% to about 90% of the composition is sublimable.

9. The composition of claim 8, wherein at least about 20%, or at least about 40%, or at least about 60%, of the composition is sublimable.

10. The composition of any one of claims 1 to 9, comprising a fragrance agent having an odor detection threshold (ODT) less than about 0.5 ppb.

11. The composition of claim 10, wherein the fragrance agent has an ODT of less than about 0.2 ppb.

12. The composition of claim 10, wherein the fragrance agent has an ODT of from 0.0001 to about 0.5 ppb.

13. The composition of any one of claims 1 to 12, comprising a fragrance agent having an average molecular weight of less than about 300 Da.

14. The composition of claim 13, wherein the fragrance agent has an average molecular weight of less than 250 Da.

15. The composition of claim 13, wherein the fragrance agent has an average molecular weight of less than 225 Da.

16. The composition of any one of claims 1 to 15, wherein the composition comprises musk oil, civet oil, ambergris oil, castoreum oil, abies oil, ajowan oil, almond oil, ambrette seed absolute, angelic root oil, anise oil, basil oil, bay oil, benzoin resinoid, bergamot oil, birch oil, bois de rose oil, broom absolute, cajeput oil, cananga oil, capsicum oil, caraway oil, cardamon oil, carrot seed oil, cassia oil, cedar leaf oil, cedar wood oil, celery seed oil, cinnamon bark oil, citronella oil, clary sage oil, clove oil, cognac oil, coriander oil, cubeb oil, cumin oil, camphor oil, dill oil, elemi gum, estragon oil, eucalyptol nat., eucalyptus oil, fennel sweet oil, galbanum res., garlic oil, geranium oil, ginger oil, hop oil, hyacinth absolute, jasmine absolute, juniper berry oil, labdanum res., lavender oil, laurel leaf oil, lemon oil, lemongrass oil, lime oil, lovage oil, mace oil, mandarin oil, mimosa absolute, myrrh absolute, mustard oil, narcissus absolute, neroli bigarade oil, nutmeg oil, oakmoss absolute, olibanum res., onion oil, opoponax res., orange oil, orange flower oil, origanum, orris concrete, pepper oil, peppermint oil, peru balsam, petitgrain oil, pine needle oil, rose absolute, rose oil, rosemary oil, safe officinalis oil, sandalwood oil, sage oil, spearmint oil, styrax oil, thyme oil, tolu balsam, tonka beans absolute, tuberose absolute, turpentine oil, vanilla beans absolute, vetiver oil, violet leaf absolute, ylang oil, α-pinene, β-pinene, d-limonene, 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol, anethole, musk xylol, isoeugenol, methyl eugenol, α-amylcinnamic aldehyde, anisaldehyde, n-butylaldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hydroxylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glicydate, methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl .alpha.-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, and vetiveryl acetate.

17. The composition of any one of claims 1 to 16, wherein one or more fragrance agents comprise terpenoid compounds.

18. The composition of claim 17, wherein the terpenoid compounds are monoterpenoids, diterpenoids, or sesquiterpenoids.

19. The composition of claim 17, wherein at least one terpenoid is 1,8-cineol, anisole, anethole, eugenol, thymol, linalool, borneol, citronellol, menthol, isomenthol, camphor, camphene, carvone, geraniol, menthone, pulegone, limonene, α-pinene, β-pinene, sabinene, myrcene, α-terpinene, β-bisabolene, trans-α-bergamotene, nerol, neral, cedrol, cedrene, thujopsene, methyl thujate, and thujic acid.

20. The composition of any one of claims 1 to 19, comprising one or more fragrance agents selected from menthol carboxamide, ethylene brassylate, methyl dihydrojasamanate, oil of lemon eucalyptus, menthol, camphor, and geraniol.

21. The composition of any one of claims 1 to 20, wherein the composition comprises one or more of DEET, soy bean oil, and menthane 3,8 diol.

22. The composition of any one of claims 1 to 21, further comprising one or more of a stabilizer, a preservative, an antioxidant, a solvent, a chelating agent, a surfactant, an antifoaming agent, a viscosity regulator, a binder, a propellant, and an encapsulating agent.

23. The composition of claim 22, wherein the composition is encapsulated in a matrix.

24. The composition of claim 23, wherein the matrix is water soluble.

25. The composition of claim 23, wherein the matrix is water insoluble.

26. The composition of claim 24, wherein the composition can be mixed with a fluid to provide a homogenous mixture with simple shaking to vigorous stirring.

27. The composition of any one of claims 1 to 26, wherein the composition is packaged as a product selected from skin care product, fabric care product, air care product, hair care product, home and/or environment care product, hygiene/beauty care product, fine fragrance, an over-the-counter health product, and a device for one or more thereof.

28. The composition of claim 22, wherein the product is used on a human body, in pet care, in indoor air, in outdoor air, or on an inanimate surface.

29. A method of repelling an insect or an arachnid, the method comprising contacting air from a space or contacting a surface with the composition of any one of claims 1 to 28.

30. The method of claim 29, wherein the space is an indoor space.

31. The method of claim 29, wherein the space is an outdoor space.

32. The method of any one of claims 29 to 31, wherein the surface is human skin, human hair, or human clothing.

33. The method of any one of claims 29 to 31, wherein the surface is animal skin or animal fur.

34. The method of any one of claims 29 to 31, wherein the surface is an inanimate surface selected from countertops, walls, floor, or fabric.

35. The method of any one of claims 29 to 34, wherein the insect is selected from the taxonomic order Blattaria, Anoplura, Sternorrhyncha, Auchenorrhyncha, Coleorrhyncha, Hymenoptera, Lepidoptera and Siphonaptera.

36. The method of claim 35, wherein the insect is selected from mosquitoes, lice, fleas, bed bugs, kissing bugs, ants, bees, wasps, flies, and sand flies.

37. The method of any one of claims 29 to 34, wherein the arachnid is from the taxonomic order Acarina selected from ticks, mites and spiders.

38. A method of reducing transmission of an infection by an insect or arachnid vector, the method comprising contacting a human or animal subject, or contacting air or surface proximal to said human or animal subject, with the composition of any one of claims 1 to 28.

39. The method of claim 38, wherein the infection is selected from an infection caused by an arbovirus, Lyme disease, bubonic plague, malaria, leishmaniasis, onchocerciasis, Chagas Disease, filariasis, Schistosomiasis, Typhus, Crimean-Congo haemorrhagic fever, Sleeping sickness (African trypanosomiasis), Louse-borne relapsing fever, Anaplasmosis, Babesiosis, Borrelia mayonii infection, Borrelia miyamotoi infection, Bourbon virus infection, Colorado tick fever, Ehrlichiosis, Heartland virus infection, Powassan disease, Rickettsia parkeri rickettsiosis, Rocky Mountain spotted fever (RMSF), STARI (Southern tick-associated rash illness), Tickborne relapsing fever (TBRF), Tularemia, and 364D rickettsiosis.

40. The method of claim 39, wherein the arbovirus is selected from Dengue virus, Zika virus, Japanese encephalitis virus, Rift Valley fever virus, Tick-borne encephalitis virus, West Nile virus and Yellow fever virus.

41. The method of any one of claims 38 to 40, wherein the vector is selected from a mosquito, a fly, a Tsetse fly, a sand fly, a louse, a flea, a tick, a mite, and a black fly.

42. The method of any one of claims 38 to 41, wherein the composition is contacted with human skin, human hair, or human clothing.

43. The method of any one of claims 38 to 41, wherein the composition is contacted with animal skin or animal fur.

44. The method of any one of claims 38 to 41, wherein the surface is an inanimate surface selected from countertops, walls, floor, or fabric.
